(19) United States
(12) Patent Application Publication
(10) Pub. No.: US 2023/0330374 A1
(43) Pub. Date: Oct. 19, 2023

(54) VENTILATOR DEVICE WITH FRAME AND DEVICE HOUSING ACCOMMODATED ROTATABLY THEREON

(71) Applicant: Hamilton Medical AG, Chur (CH)

(72) Inventor: Daniel Barandun, Chur (CH)

(21) Appl. No.: 18/026,453

(22) PCT Filed: Sep. 22, 2021
(86) PCT No.: PCT/EP2021/076055

§ 371 Date: Mar. 15, 2023

(30) Foreign Application Priority Data
Sep. 24, 2020 (DE) 10 2020 124 978.0

(51) Int. Cl. A61M 16/00

(52) U.S. Cl. CPC A61M 16/024; A61M 16/0066

(57) ABSTRACT

A ventilator device for at least supporting artificial ventilation of patients, including:
a device housing,
a functional arrangement received in the device housing, the functional arrangement including, as functional units, at least one portion of a breathing gas line, a pressure-changing device for changing a breathing gas pressure in the breathing gas line, and a control device for controlling the operation of at least one pressure-changing device, and
an input/output device, which is arranged on the device housing, is accessible from outside the device housing in order to be operated, and is intended for inputting data and/or control commands at the control device and/or for outputting data and information, the input/output device being connected to the control device for signal transmission; the ventilator device having a frame, the device housing with the input/output device arranged thereon being received on the frame so as to be rotatable about a virtual rotation axis.

This application claims priority in PCT application PCT/EP2021/076055 filed on Sep. 22, 2021, which claims priority in German Patent Application DE 10 2020 124 978.0 filed on Sep. 24, 2020, which are incorporated by reference herein.

The present invention concerns a ventilator device for at least supportive artificial respiration of patients. The ventilator device comprises:
A device housing,
A functional arrangement accommodated in the device housing, which as functional units exhibits at least a section of a respiratory gas line, a pressure modification device for modifying a respiratory gas pressure in the respiratory gas line, and a control device for controlling the operation at least of the pressure modification device, and
An input/output device arranged at the device housing, accessible from outside the device housing for its operation, for the input of data and/or control commands into the control device and/or for the output of data and information, where the input/output device is linked with the control device for signal transmission.

Preferably the ventilator device is an emergency ventilator device as used by emergency physicians and first aiders in emergency situations.

### BACKGROUND OF THE INVENTION

As such an emergency ventilator device there are known the ventilator device with the designation ‘EVE_{IN}’ of Fritz Stephan GmbH in Gackenbach (DE) and the ventilator device with the designation ‘Falco 202 Evo’ of the Italian firm Siare Engineering International Group s. r. l. in Valsamoggia (IT).

As a portable medical device, albeit not a ventilator device, there is further known a defibrillator under the type designation ‘corpuls³’ of the firm GS Electromedizinische Geräte G. Stemple GmbH in Kaufering (DE). In an operational configuration, the known defibrillator is pivotable over an angular range of approximately 30° in a frame which accommodates the defibrillator.

Moreover, there is known the ventilator ‘Hamilton-G5’ of the applicant, which exhibits a monitor with an operator's console which is pivotable relative to the rest of the device housing.

Emergency ventilator devices, inter alia also referred to as ‘intensive care ventilators’, serve for rapid supply of respiratory gas to a patient outside a clinical environment, i.e. for instance at an accident site and/or during transportation of a patient. Of course, emergency ventilator devices can also be used in a clinical environment, however in hospitals often more powerful ventilators are available as emergency ventilator devices.

As ventilator devices which are deployable outside a clinical environment, emergency ventilator devices, such as preferably also the present ventilator device, exhibit their own energy store which at least for a certain duration allows operation of the emergency ventilator device independently of a power grid supply. Furthermore, emergency ventilator devices, like also preferably the present ventilator device, are designed as portable ventilator devices with regards to their size and weight, such that they can be moved by emergency medical personnel, for instance an emergency physician called to an accident site, with just their own muscle strength even over distances of several dozen meters without excessive physical stress.

Where emergency ventilator devices exhibit a fan as a preferred pressure modification device, these emergency ventilator devices can administer at least ambient air as respiratory gas without supplementary special gas reservoirs, such as for instance a detachably coupled oxygen reservoir. To the ambient air there can be added where needed a special gas different from the ambient air, in the most frequent case pure oxygen but also anesthetic and/or therapeutic gases and gas mixtures. To this end, emergency ventilator devices, such as also those of the present invention, usually exhibit a connector formation for connecting a special gas reservoir.

In order to protect the functional arrangement from external influences, such as mechanical impact, dirt, and the like, it is arranged in the device housing.

For the input/output device to make possible operation of the ventilator device and the observing of information about an operational state of the ventilator device and/or a respiratory state of a patient ventilated through the ventilator device, it is arranged at the device housing, namely in such a manner that it is accessible to an operator from outside the device housing. Through the signal-transmitting linking of the input/output device and the control device, signals and consequently data and information can be transmitted between these devices.

Due to the ability of the present ventilator device to be deployable as a portable ventilator device at accident sites and in in medical transport vehicles, including ground-based vehicles, aircraft, and watercraft, it is important for the ventilator device to be adaptable to different operational environments and operational situations and to be optimally operable in these different operational environments and situations. Only in the smallest number of cases is an ergonomically suitable supporting surface available as a working surface for the ventilator device.

### SUMMARY OF THE INVENTION

It is, therefore, the task of the present invention to improve the ventilator device as mentioned at the beginning in such a way that it can be deployed and optimally operated even in operational environments which are not prepared for its deployment.

The present invention solves this task through a ventilator device of the type mentioned at the beginning which exhibits a frame where the device housing with the input/output device arranged thereon is accommodated at the frame rotatably about a virtual axis of rotation. The frame allows in principle solid placement of the ventilator device at the respective deployment site. Through the rotatable accommodation about an axis of rotation of the device housing together with the input/output device arranged thereon, the overall arrangement consisting of device housing and input/output device can be rotated after placement of the frame into an orientation in which the overall arrangement for the artificial respiration of a patient is both operational and can be operated as conveniently as possible. In this way the ventilator device with the frame can be set down on the ground of an accident site, or at a distance from the ground on the hood of a vehicle, or at a setting-down place available at the respective deployment site. The impact of the different operating situations, effected first and foremost by the different possible setting-down heights, on the person operating the ventilator device can be mitigated through the rotatable accommodation of the device housing in the frame. Thus the device housing with the input/output device can be rotated in the frame about the axis of rotation in such a way that the input/output device faces towards the person operating it.

The rotatability of the overall arrangement consisting of device housing and input/output device is preferable to the known relative tiltability of an operator's console or of a control panel as an input/output device relative to the rest of the device housing, since the ventilator device remains compact regardless of the orientation of the overall arrangement. Were, instead, only the input/output device to be folded out away from the device housing towards the operating person, the risk would be increased of other persons moving about the operational vicinity of the ventilator device colliding with the folded out device part and thereby impairing the ventilation situation of an artificially respirated patient. Here it should ultimately be taken into account that first and foremost at accident sites there prevails hectic activity of various and in part non-coordinated rescue personnel, such as emergency physicians and paramedics, firefighters and police and possibly civilian first-aiders, of which the majority might overlook the deployed ventilator device when focusing on their respective task. Therefore, the more compact the ventilator device, the more secure its operation even under panic-like exceptional conditions.

Although it should not be ruled out that the input/output device can be movable relative to the device housing, nevertheless preferably the device housing is accommodated together with the input/output device rotatably about the axis of rotation in the frame. Preferably the input/output device is arranged in the latter rigidly, i.e. immovably relative to the device housing.

In forming the most compact possible device housing, which with full functionality takes up the smallest possible spatial volume, it has proved advantageous if the device housing exhibits a prism shape extending along a prism axis. Such a prism shape comprises a housing casing wall encircling the prism axis at a radial distance from the prism axis. The axis of rotation preferably proceeds collinearly with the prism axis or at a distance from the prism axis in parallel to it. This has the advantage that the volume traversed by the device housing with the input/output device as a movement space during rotation about the axis of rotation is small compared with other relative orientations of the axis of rotation and the prism axis. Thereby too, there is reduced the collision risk with the ventilator device of persons moving about the deployment site of the ventilator device without paying attention to the ventilator device.

The frame can define at least one supporting surface, for instance through at least one supporting formation, such as for example a plurality of supporting projections, which are configured for contact with a setting-down surface when setting down the frame. Preferably the at least one supporting formation defines at least one virtual supporting surface, which for example can be a supporting surface abutting tangentially against the at least one supporting formation. When setting down the frame, the supporting surface of the ventilator device and the setting-down surface of the device's neighborhood then coincide. Alternatively or additionally, the axis of rotation can be parallel to the supporting surface. Preferably the supporting surface is a supporting plane. Preferably the at least one supporting formation defines a plurality of virtual supporting surfaces, whose position and/or orientation relative to one another can be varied by rotating the device housing about the axis of rotation in order to offer an operating person several options for setting down the ventilator device.

Preferably the axis of rotation proceeds at a distance from at least one output surface, such as for instance monitor, of the input/output device, in particular from the input/output device, such that when rotating the overall arrangement about the axis of rotation the output surface and/or the input/output device can travel through a relatively long curved path. Consequently, by rotating the overall arrangement in the frame relative to the person operating the ventilator device, the input/output device can, to a considerable extent, be displaced and at the same time be angularly oriented.

In principle, the axis of rotation can proceed outside the device housing, which leads to a relatively large movement space of the device housing with the input/output device. A compact movement space with nevertheless adequate possibility for orienting the overall arrangement in accordance with its setting-down situation can be obtained by having the axis of rotation penetrating through the device housing.

The ventilator device normally exhibits a plurality of physical functional interfaces. These include, for example, a respiratory gas aspiration aperture and/or a connector for establishing an electrical connection and/or a connector for establishing a fluid-mechanical connection and/or a connector for establishing a mechanical connection and so forth.

Preferably the ventilator device comprises a fan as the pressure modification device mentioned at the beginning, such that the ventilator device can aspirate ambient air as so to speak unlimited available respiratory gas through a respiratory gas aspiration aperture and convey it to the patient.

A functional interface for establishing an electrical connection can serve to supply the ventilator device with electrical energy or can serve to connect external peripheral devices, such as for example a printer or a modem or a data store.

A functional interface for establishing a fluid-mechanical connection can alternatively or additionally to the fan serve to connect a respiratory gas reservoir or can serve to connect a special gas reservoir, for instance oxygen or laughing gas, which is to be added to the respiratory. The aforementioned connector formation, for instance a fluid-mechanical quick coupling, can be such a functional interface. A functional interface for establishing a fluid-mechanical connection can besides serve to connect a ventilation hose, via which respiratory gas from the pressure modification device in the device housing is conveyed to a patient.

A functional interface for establishing a mechanical connection can serve to connect an aspiration-side filter which has specific filter properties extending beyond the filter properties of a respiratory gas filter preferably arranged interchangeably in the device housing.

According to the present invention, a gas reservoir can be a gas reservoir accommodated in a container or a gas reservoir provided in a building or a transport vehicle for connection by means of installation lines and coupling formations.

Since during the operation of the ventilator device there are connected to at least a part of the functional interfaces provided at the device housing lines which proceed away from the device housing, to prevent unnecessarily large-scale line movements through rotation of the overall arrangement about the axis of rotation the plurality of functional interfaces are arranged in at least one section of the device housing facing predominantly or even exclusively along the axis of rotation.

In the case of the preferred prism-shaped device housing, the plurality of functional interfaces are arranged on at least one axially with respect to the prism axis end-side front face of the device housing. For the aforementioned reason, especially preferably all the functional interfaces provided at the device housing are arranged in a section of the device housing facing along the axis of rotation and/or the prism axis respectively, in particular on an axially end-side front face of the device housing.

In the preferred case of a fan as the pressure modification device, functional interfaces are preferably arranged distributed at both axially end-side front faces of the device housing, such that respiratory gas can be aspirated by the fan at a front face into the device housing and conveyed at the axially opposite front face out of the device housing towards the patient.

Preferably the plurality of physical functional interfaces, especially preferably all the physical functional interfaces of the device housing, are configured and arranged at the device housing in such a way that with respect to the axis of rotation they face in the axial direction and/or are connectable with a counter-interface through a connecting movement which takes place in the axial direction. Then rotation of the overall arrangement about the axis of rotation results in only a small change both in the location of a functional interface and in the trajectory for establishing a connection with it. This considerably facilitates the handling and the operation of the ventilator device in a hectic environment.

In the preferred case of a prism-shaped device housing with an axis of rotation parallel to or coaxial with the prism axis, the plurality of physical functional interfaces, especially preferably all the physical functional interfaces of the device housing, are arranged on an axially end-side front face of the device housing in the aforementioned manner.

In principle it is conceivable to mount the device housing directly rotatably on the frame, for instance by means of a slide-bearing pairing, whose one slide bearing part is a slide bearing formation on the frame and whose other slide bearing part is a slide bearing counter-formation on the device housing. It can, however, be advantageous to couple the device housing detachably as specified with the frame. ‘Detachably as specified’ means, in terms of the present application, that the device housing can, at a reasonable assembly cost, be coupled with the frame non-destructively and removed again from the frame.

Preferably, therefore, the frame exhibits a first frame section holding the device housing and a second frame section holding the first frame section, where the first frame section is mounted on the second frame section rotatably about the axis of rotation. Consequently the rotary mounting can be configured between the first and the second frame section separately and independently from the device housing and its shape. The device housing and with it the input/output device arranged thereon can therefore be free from functional components of the rotary mounting.

According to a preferred further development of the present invention, the frame, in particular the previously mentioned second frame section, exhibits locking device for locking the overall arrangement in a rotational position onto the frame. A device consisting of the locking device on the one hand and a device consisting of the overall arrangement and/or the first frame section or the second frame section and movable relative to the locking device about the axis of rotation on the other can exhibit at least one recess with which a projection of the respective other device can engage with positive locking to establish a locking engagement, in order thus to fix the overall arrangement, in particular the first frame section and the second frame section, in a relative rotational position about the axis of rotation. Preferably the locking device is arranged in an actuatable manner at the frame, especially preferably at the second frame section. Then the overall arrangement and, if present, the first frame section, are rotatable about the axis of rotation relative to the locking device.

The locking engagement of the recess and the projection is released by actuating an operating formation of the locking device, for instance by tilting at least one lever about a locking axis or by pressing a locking button. To prevent undesirable twisting of the overall arrangement about the axis of rotation, the locking device is preferably pre-tensioned in the direction of a locking engagement, for instance through a spring arrangement. Instead of a positive-locking engagement, the locking engagement can also be a frictional engagement. Then the overall arrangement could be displaced continuously about the axis of rotation relative to the frame, in particular to the second frame section, and stopped. Because of the more secure locking of the overall arrangement in a rotational position, a positive-locking engagement is preferred as a locking engagement.

Preferably both the first frame section and the second frame section can each form a part of the supporting formation on which the ventilator device can be set down on a footing. Preferably the first frame section exhibits at least one, preferably at least two, supporting projections, which are configured for standing up on a footing. Preferably the second frame section likewise exhibits at least one, preferably at least two supporting projections, such that the supporting projections of the first and of the second frame section together can form at least one three-point supporting formation on which the ventilator device can be securely set down on a footing regardless of the respective rotational position of the overall arrangement relative to the frame. In order to prevent tilting about a tilting axis connecting two of the three supporting points which is typical for setting up on three points, the first and the second frame section can jointly form a four-point supporting formation, where preferably two supporting points are formed by the first frame section and a further two supporting points by the second frame section, in order to be able again to achieve positioning of the ventilator device on an even footing regardless of the respective rotational position of the overall arrangement.

The first frame section can exhibit more than two supporting projections, such that the ventilator device in one and the same relative position of the frame sections to each other can either be set down on a footing by means of a supporting formation formed by supporting points of the first and the second frame section or can be set down on a footing by means of a supporting formation formed by supporting points only of the first frame section. In principle it is likewise conceivable for the second frame section to exhibit more than two supporting projections. Then the ventilator device can be set down on a footing by means of a supporting formation formed by supporting points only of the second frame section.

In certain operational cases, a compact ventilator device requiring as little installation space as possible can be more advantageous than a ventilator device with an overall arrangement consisting of device housing and input/output device which is rotatable about an axis of rotation relative to a frame. For this case it can be provided that the first frame section is accommodated detachably as specified at the second frame section. Consequently the device housing can either be used without a frame, or the device housing can be used without the first frame section, or the device housing can be used without the second frame section.

In order to make sure that both frame sections are each connectable on its own with the device housing, it is advantageous if both the first frame section and the second frame section each exhibit an attachment formation which is configured to interact with an attachment counter-formation arranged or configured at the device housing for attachment to the respective frame section. Then the device housing can be attached solely to the first frame section, which in turn is connected with the second frame section rotatably about the axis of rotation relative to the second frame section. Or the device housing can be attached solely to the second frame section, where it is then immovable relative to the second frame section. The second frame section can then serve as a carrier frame.

The attachment of the device housing to a frame section out of the first and second frame section can be realized through a clamped connection or latching connection. To this end, at the first and/or the second frame section there can be provided a clamping formation which clamps a section of the device housing. Or a latching formation can be provided which can be brought into a latching engagement with a latching counter-section of the device housing.

When each of the frame sections is to be directly connectable with the device housing, a bolted connection is advantageous for reasons of the advantageously small installation space required for this with at the same time high connection security. For example, for this purpose each of the frame sections can exhibit a predetermined number of clearance holes and the device housing can exhibit threaded holes which in a predetermined attachment position of the device housing relative to the respective frame section align with its clearance holes.

In order to facilitate the carrying of the ventilator device, a carrying handle can in principle be provided at the device housing. If, however, the device housing is in any case configured for use with the frame, it is more advantageous to configure the device housing as compactly as possible and a carrying handle configured for manual handling at the frame.

For operating the ventilator, in particular for orienting the input/output device about the axis of rotation, it is advantageous if the carrying handle runs parallel to the axis of rotation.

In particular for secure carriage of the ventilator device in emergency medical transport vehicles, the ventilator device can exhibit a suspension arrangement which is configured to fix the ventilator device detachably as specified onto an external structure. The term ‘transport vehicle’ comprises here ground-based vehicles, in particular motorized vehicles, aircraft, and watercraft. It is precisely emergency medical transport vehicles which in the vicinity of the patient transported by them normally exhibit scarcely any setting-down surfaces which would allow the setting down of the ventilator device. Patient transport spaces of such transport vehicles are normally maximally utilized for accommodating their own on-board emergency medical equipment and storage spaces. Through the suspension arrangement, however, a simple external structure in the transport vehicle or also at the deployment location at an accident site suffices for attaching the ventilator device to the structure in the event that setting down the ventilator device is not possible due to the nature of the footing or the lack of adequate setting-down space. The present invention therefore concerns also a ventilator device of the type mentioned at the beginning, which exhibits a suspension arrangement for detachable fixing of at least one overall arrangement consisting of device housing and input/output device to an external structure. The ventilator device with suspension arrangement can exhibit a frame at which the device housing is accommodated. The ventilator device provided with a suspension arrangement does not necessarily have to be accommodated at the frame rotatably about an axis of rotation, although this is preferable. Further developments of the frame which are described above which do not concern the rotatability of the overall arrangement about the axis of rotation are also further developments of the ventilator device with frame and suspension arrangement in which the overall arrangement is accommodated non-rotatably at the frame.

The suspension arrangement can comprise a loop or lug. Then the external structure can comprise or be a hook and/or a projection. Preferably the suspension arrangement comprises at least one hook or is a hook, such that a simple loop or lug or even only a rod or bar suffices as an external structure for suspended attachment of the ventilator device.

In order to be able to suspend the ventilator device from as many different external structures as possible, according to a preferred further development of the present invention the suspension arrangement is movable relative to the device housing and/or relative to the frame. Then the suspension arrangement, in particular the hook, can be brought into a position relative to the frame which is advantageous for attaching the ventilator device for the respective external structure encountered. Preferably the suspension structure is rotatable about a suspension axis. Preferably the suspension axis here is oriented in parallel to the axis of rotation, in order to be able to ensure, after suspending the ventilator device from an external structure, the most advantageous and simple handling possible of the ventilator device in the position then reached of the overall arrangement through rotation of the overall arrangement about the axis of rotation.

Especially preferably, the suspension arrangement comprises a plurality of hooks. Preferably the suspension axis runs through the carrying handle configured for manual handling at the frame, where the carrying handle itself can be configured firmly as a rod or rod section. According to an especially preferred embodiment form, which makes possible a stable suspension of the ventilator device without a tendency to oscillate, there is a hook arranged at each of the two longitudinal ends of the carrying handle. For simple, synchronized rotating of the plurality of hooks, the carrying handle is preferably a shaft connecting at least two hooks, especially preferably the two hooks arranged at the longitudinal ends of the carrying handle.

The suspension arrangement is preferably lockable in different relative positions relative to the frame carrying it, in order to be able to secure the ventilator to the external structure in a position once attained.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1A rough schematic depiction of an embodiment form according to the invention of a ventilator device of the present invention,

FIG. 2A rough schematic depiction of the embodiment form according to the invention of the ventilator device of FIG. 1 with the overall arrangement consisting of device housing and input/output device rotated about an axis of rotation compared with FIG. 1,

FIG. 3A rough schematic depiction of the embodiment form according to the invention of the ventilator device of FIGS. 1 and 2 when observed along the axis of rotation, and

FIG. 4A rough schematic depiction of the embodiment form according to the invention of the ventilator device of FIGS. 1 to 3 with suspension device.

### DESCRIPTION OF PREFERRED EMBODIMENTS

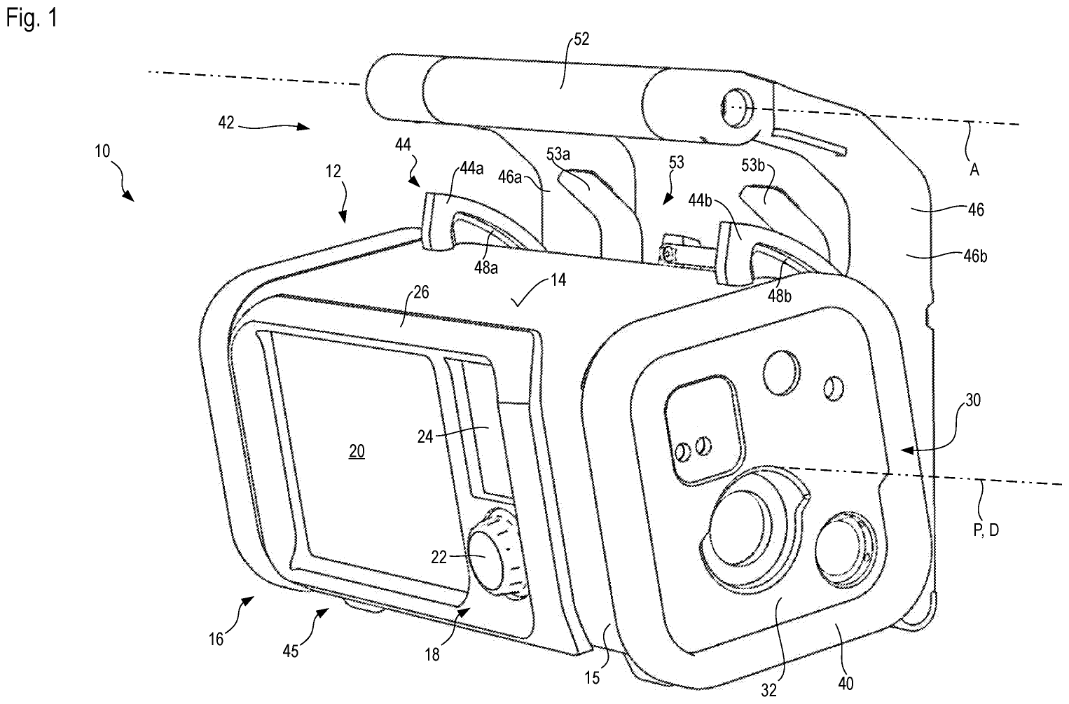

Referring now to the drawings wherein the showings are for the purpose of illustrating preferred and alternative embodiments of the invention only and not for the purpose of limiting the same, in FIG. 1, an embodiment form according to the invention of a ventilator device of the present application in a preferred embodiment as an emergency ventilator is labelled generally by 10. The emergency ventilator 10 comprises a device housing 12 with a prismatic basic form, in the present case with a cuboidal basic form with rounded edges.

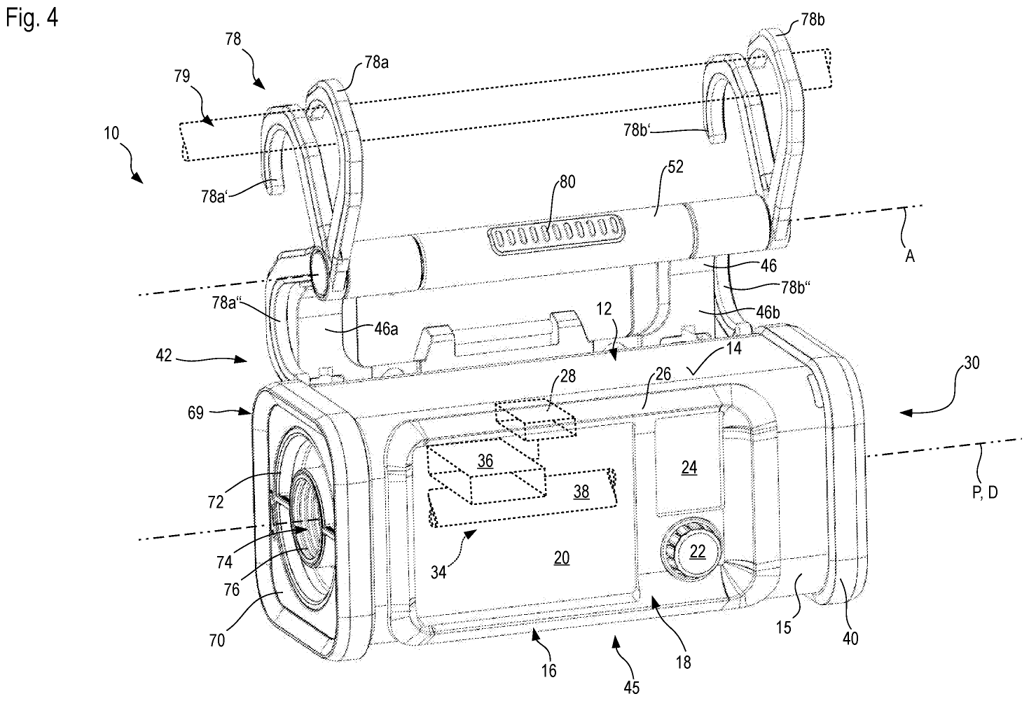

The device housing 12 extends along a virtual prism axis P, which is conceived as penetrating through the device housing 12 centrally along its longitudinal dimension. Encircling the prism axis P at a radial distance from the latter, there extends a housing casing surface 14 in which a structural unit 16 with an input/output device 18 is accommodated. The input/output device 18 comprises a monitor 20 for visual output of data and information. The monitor 20 is preferably a touchscreen, such that via the monitor 20 data and control commands can also be input into the input/output device 18. At the input/output device 18 there is furthermore depicted a rotary switch 22 and a keypad 24 for the input of data and/or control commands indicated. The mentioned switches are only chosen by way of example. A border 26 overlaps an edge of the housing casing surface 14 in order to seal an aperture in the housing casing surface 14 which is penetrated through by the structural unit 16 of the input/output device 18 and bordered by the edge of the housing casing surface 14. Through the aperture penetrated through by the structural unit 16, the input/output device 18 is connected for signal transmission with a control device 28 arranged inside the device housing 12 (see FIG. 4).

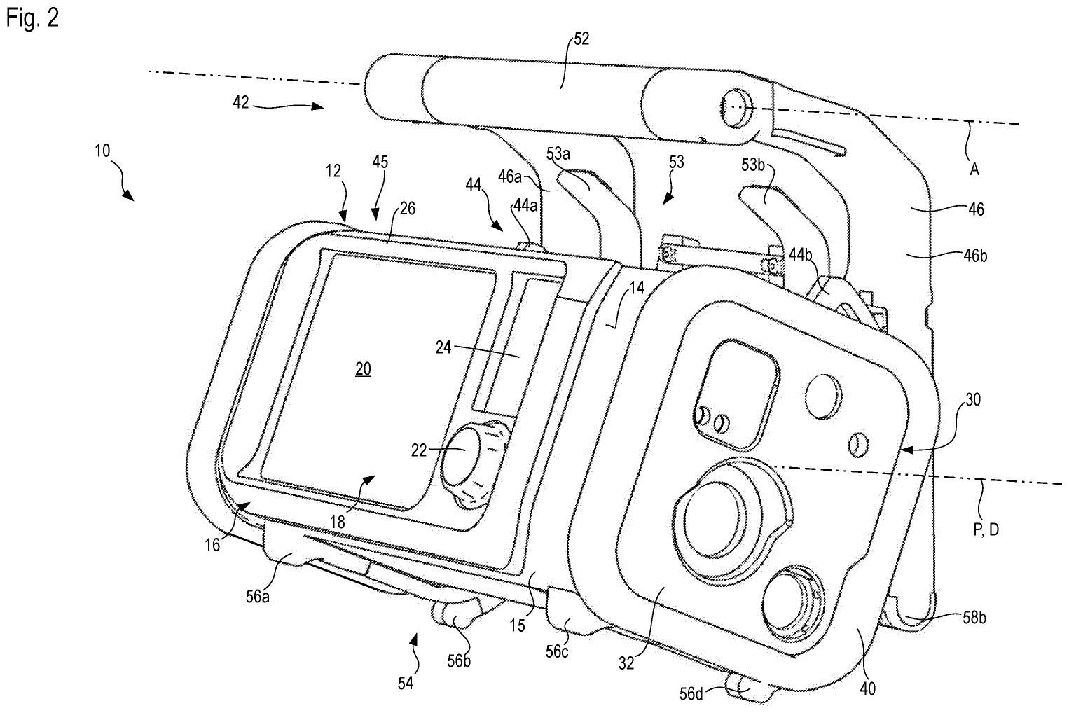

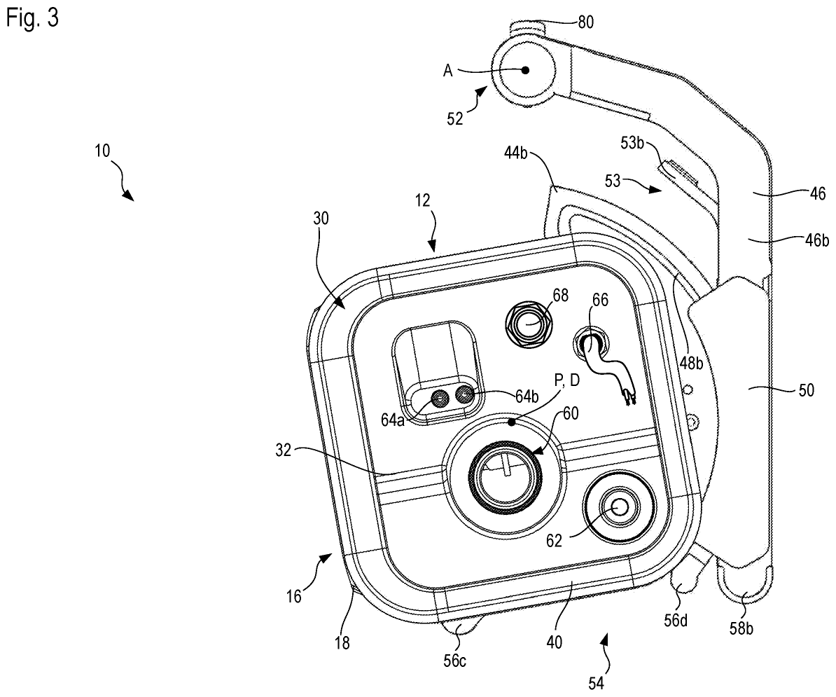

The observer of FIG. 1 also looks at a right front face 30 as seen by a person operating the input/output device 18, which exhibits various surface areas for accommodating functional interfaces elucidated in more detail further below in connection with FIG. 3. In FIGS. 1 and 2 the functional interfaces are identified merely by placeholders.

The housing casing surface 14 is formed by an extruded or metal-formed tube component 15, in particular made from aluminum for better heat conduction.

The front face 30 comprises a cover plate 32, which preferably is part of a functional arrangement 34 (see FIG. 4) to which the control device 28, a fan 36 as a pressure modification device, and a respiratory gas line 38 belong. The control device 28 is connected with the fan 36 for signal transmission, such that the control device 28 can control the operation of the fan 36. The functional arrangement 34 is preferably a preassembled module which is introduced as a whole into the tube component 14 along the prism axis P. The functional arrangement 34 can comprise further functional units which are not depicted in the present case but only mentioned, such as for example a respiratory gas filter, a power supply unit for transforming an external electric voltage to the unit voltage of the ventilator device 10, an accommodating duct for an electric energy store and/or the electric energy store, a cooling element for dissipating heat from the fan 36 to the housing casing surface 14, and the like.

In the region where the tube component 15 and the cover plate 32 meet there is arranged an encircling elastomer buffer 40 as edge protection for the housing edge formed by the tube component 15 and the cover plate 32. The elastomer buffer 40 can be injection-molded from a thermoplastic elastomer. It can be made from silicone rubber, natural rubber, or synthetic rubber.

The ventilator device 10 further comprises a frame 42, comprising a first frame section 44 which is connected directly with the overall arrangement 45 consisting of device housing 12 and input/output device 18, for instance by bolting. The bolting or generally the connection with the first frame section 44 is preferably made on the back of the device housing 12 which is opposite the housing front with the input/output device 18.

The frame 42 further comprises a second frame section 46, at which the first frame section 44 is arranged guided rotatably about an axis of rotation D. The first frame section 44 with the overall arrangement 45 fixed thereon is rotatable about the axis of rotation D over an angular region of at least 40°, preferably of at least 50°. In the present example, the axis of rotation D coincides collinearly with the prism axis P. Preferably, the axis of rotation D does not penetrate through the frame 42.

The first frame section 44 exhibits two arms 44a and 44b curved in a part-circular shape at least along their guiding regions, which encompass a section of the device housing 12 in the circumferential direction about the prism axis P and which each exhibit a part-circular profile formation 48a or 48b respectively, which are guided displaceably in a translatory manner in a guide component 50 (see FIG. 3) along the part-circular shape of the arms 44a and 44b along a part-circular path about the axis of rotation D, with the axis of rotation D as the center point axis of the part-circular path. The profile formations 48a and 48b can be a T-shaped, an L-shaped, or a double T-shaped profile, to mention only a few examples.

The second frame section 46 exhibits two parallel arms 46a and 46b, at whose longitudinal ends distant from the device housing 12 there is arranged a carrying handle 52 connecting the arms 46a and 46b. As will be elucidated further below in connection with the preferred further development of the ventilator device 10 of FIG. 4, the carrying handle 52 can be arranged rotatably relative to the arms 46a and 46b about a suspension axis A. The suspension axis A is preferably parallel to the axis of rotation D. The carrying handle 52, when it is mounted rotatably about the suspension axis A, is preferably cylindrical such that its outer surface is largely or completely invariant under a rotation about the suspension axis A.

The arms 46a and 46b are configured curved about an axis of curvature which is parallel to the axis of rotation D and/or prism axis P respectively, namely preferably such that regardless of the rotational position of the overall arrangement 45 the arms 46a and 46b partly encompass the overall arrangement 45 about the axis of rotation D in the circumferential direction. The total mass of the ventilator device 10 is here balanced in such a way and/or the handle 52 is positioned in such a way respectively, that when the ventilator device 10 is suspended freely from the handle 52 with a suspension axis A orthogonal to the gravitational direction, then regardless of the respective rotational position of the overall arrangement 45 relative to the housing 42, a connecting plane which contains both the suspension axis A and the prism axis P is inclined with respect to the gravitational direction in absolute terms by no more than 10°, preferably by no more than 7°.

FIG. 2 depicts again the ventilator device 10 of FIG. 1, where however the overall arrangement 45 is rotated into another position clockwise about the axis of rotation D.

The position of the overall arrangement 45 in the frame 42 in FIG. 1 can for example be chosen when the ventilator device 10 is arranged at a relatively great height, for instance above head height of a person operating the ventilator device 10. The overall arrangement 45 can then be so oriented that the operating section of the input/output device 18 with the monitor 20, the switch 22, and the keypad 24 faces downwards away from the carrying handle 52. In contrast to this, the position of the overall arrangement in the frame 42 of FIG. 2 can be chosen when the ventilator device 10 is arranged relatively low, for instance on a ground on which the person operating the ventilator device 10 is standing. Then the overall arrangement 45 can be so oriented that the operating section of the input/output device 18 faces upwards towards the carrying handle 52.

The frame 42, in the presented embodiment example in particular the second frame section 46, exhibits a locking device 53 for locking the overall arrangement 45 in a rotational position. Of the locking device 53 there are depicted in FIGS. 1 to 3 two levers 53a and 53b which can be tilted together about a locking axis which is preferably parallel to the axis of rotation D and/or to the arrangement axis A. Additionally or alternatively, the levers 53a and 53b can be displaceable in a translatory manner. A device consisting of the locking device 53 and the first frame section 44, in particular the part-circular profile formation 48a or 48b as the case may be, can exhibit at least one recess with which a projection of the respective other device can engage with positive locking in order thus to fix the first frame section 44 and the second frame section 46 in a relative rotational position about the axis of rotation D. The locking engagement is released by tilting the levers 53a and/or 53b about the locking axis. To prevent undesirable twisting of the overall arrangement 45 about the axis of rotation D relative to the second frame section 46, the locking device 53 is pre-tensioned in the direction of a locking engagement. Instead of a positive-locking engagement, the locking engagement can also be a frictional engagement. Then the overall arrangement 45 could be continuously adjusted about the axis of rotation D relative to the second frame section 46 and stopped. The positive-locking engagement is preferable as a locking engagement because of the more secure locking of the overall arrangement 45 in a rotational position.

As can be best discerned in FIG. 3, but also in FIG. 2, the frame 42 exhibits a supporting formation 54 which in the depicted example exhibits four supporting projections 56a, b, c, and d, of which the supporting projections 56a and 56b are configured at the arm 44a of the first frame section 44 and the supporting projections 56c and 56d at the parallel arm 44b of the first frame section 44. Furthermore, the supporting formation 54 exhibits a supporting projection 58a configured at the arm 46a of the second frame section 46 and a supporting projection 58b configured at the arm 46b of the second frame section 46. The supporting projections 58a and 58b can also be replaced by a single supporting projection extending over the entire length of the second frame section 46 along the axis of rotation D.

The supporting projections 56a, b, c, and d of the first frame section 44 protruding from the underside of the device housing 12 do not change their relative position to one another. Likewise, the supporting projections 58a and 58b of the second frame section 46 do not change their relative position to one another. However, a twisting of the overall arrangement 45 about the axis of rotation D changes the relative position of the supporting projections 56a, b, c, and d of the first frame section 44 relative to the supporting projections 58a and 58b of second frame section 46.

In FIG. 3 it can be clearly discerned that depending on the rotational position of the overall arrangement 45 and thereby of the first frame section 44 relative to the second frame section 46, the ventilator device 10 can be set down either on a supporting surface defined only by the supporting projections 56a, b, c, and d of the first frame section 44 or on a supporting surface defined only by the supporting projections 56b and 56d of the first frame section 44 and the supporting projections 58a and 58b of the second frame section 46 or on a supporting surface defined only by the supporting projections 56a and 56c of the first frame section 44 and the supporting projections 58a and 58b of the second frame section 46.

FIG. 3 shows as functional interfaces by way of example a respiratory gas outlet aperture 60, through which inspiratory respiratory gas conveyed by the fan 36 of the pressure modification device exits from the device housing 12 towards a patient connected to the emergency ventilator 10. The respiratory gas outlet aperture 60 forms one end of the respiratory gas line 38 of the functional arrangement 34. To a special gas coupling section 62 there can be connected a supply device or a special gas reservoir to supply into the respiratory gas line 38 a gas which differs from the air aspirated by the fan as respiratory gas as an inspiratory respiratory gas or as a component of same.

FIG. 3 shows as further functional interfaces connection nozzles 64a and 64b to which pressure acquisition hoses are connectable which at their other end lying distally from the connection nozzles 64a or 64b respectively are each connected with an inner region of a differential pressure flow sensor for measuring a proximal inspiratory and preferably also expiratory respiratory gas flow. The two inner regions of the differential pressure flow sensor are separated from one another in a manner known per se through a flow resistance which is variable through the respiratory gas flow.

Given spatial availability of a power connection, the emergency ventilator 10 can be operated via a power input 66 with energy from an external power supply grid such as for instance from a public power supply grid or from the on-board supply grid of a vehicle. All electrical functional units of the emergency ventilator 10 can then be supplied with grid energy, where the grid voltage is transformed to DC low voltage via a power supply unit as a further functional unit of the preassembled functional arrangement 34. Likewise, an undepicted storage battery can be recharged. A socket 68 as a further functional interface in the housing 12 is arranged for connecting an external sensor, in particular CO₂ sensor. Such a CO₂ sensor can for example be provided at a flow sensor coupled with the emergency ventilator device 10 and be coupled to a sensor arrangement.

Through the arrangement of the functional interfaces in the cover plate 32, the displacement paths of the individual functional interfaces under rotation of the overall arrangement 45 about the axis of rotation D are short. In addition, all functional interfaces are connectable with the respective assigned cable through a connecting movement which is axial with respect to the axis of rotation D of a counter-interface which is to be connected with the respective functional interface, for instance a plug or socket. Consequently, the orientation in space of a cable provided for establishing a connection with a functional interface in the cover plate 32 does not change under rotation of the device housing 12 about the axis of rotation D or only a little.

As for the rest, cables connected to functional interfaces go away from the respective functional interface in parallel to the axis of rotation D, such that a rotation of the device housing 12 about the axis of rotation D by the possible 40° to 50° does not lead to an undesirable tangle of cables, as could be the case with cables going away radially from the housing casing surface 14. A collision of the connected cables with the frame 42 is likewise nearly ruled out.

In FIG. 4 there is depicted an advantageous further developed version of the ventilator device 10 of FIGS. 1 to 3 in roughly schematic perspective. The observer of FIG. 4 also looks at the other front face 69 of the device housing 12 opposite to the front face 30, which is formed by a cover 70 that is lockable and unlockable by a rotary safety device 72 on the tube component 15. In the unlocked state, the cover 70 can be removed from the tube component 15 along the axis of rotation D.

Penetrated through centrally by the prism axis P and the axis of rotation D, the cover 70 exhibits a respiratory gas aspiration aperture 74 through which the fan 36 aspirates ambient air as respiratory gas. Preferably the aspirated ambient air is drawn in and at the same time cleaned by an undepicted filter arranged downstream from the respiratory gas aspiration aperture 74. The inner surface of the respiratory gas aspiration aperture 74 exhibits an inner thread 76 at which a mechanical object, such as for instance an additional high-performance filter, can be coupled detachably with the ventilator device 10. The respiratory gas aspiration aperture 74 and the inner thread 76 form further functional interfaces of the ventilator device. Once again these functional interfaces face in a direction which is parallel to the axis of rotation D. The thread axis of the inner thread 76 is collinear with the central axis of the respiratory gas aspiration aperture 74 and is collinear with the axis of rotation D.

The ventilator device 10 exhibits in FIG. 4 a suspension arrangement 78 with which the frame 42 with the overall arrangement 45 accommodated therein can be accommodated at an external structure 79, such as for example a rod.

The suspension arrangement 78 exhibits in the depicted example two hooks 78a and 78b. In the present example the carrying handle 52 is arranged between the two hooks 78a and 78b and is connected rigidly with the hooks 78a and 78b for common rotary movement about the suspension axis A. The carrying handle 52 and the hooks 78a and 78b are therefore rotatable relative to the arms 46a and 46b of the second frame section 46 about the suspension axis A.

Each of the hooks 78a and 78b is shown in FIG. 4 in three different positions. Positions of the hooks 78a and 78b which belong together are labelled with single and double quotation marks.

A release button 80 serves for disengaging a latching of the suspension arrangement 78 together with the carrying handle 52 to the arms 46a and 46b of the second frame section 46. The latching can be stepped latching provided with a preset division, for instance when the latching device effecting it uses a positive-locking engagement for latching. The latching device can latch continuously if it uses frictional engagement. With a non-pushed, non-pressed release button 80, the latching device of the jointly movable structural unit consisting of suspension arrangement 78 and carrying handle 52 is pre-tensioned by at least one spring arrangement in a latching position which latches the structural unit. Through the axially central arrangement of the release button 80 which is actuatable radially with respect to the arrangement axis A relative to the carrying handle 52, the hooks 78a and 78b can be rotated equally single-handedly both by left-handed and by right-handed persons about the arrangement axis A.

Alternatively to the depiction of FIG. 4, the two hooks 78a and 78b can be rotatable about the suspension axis A together but independently from the carrying handle 52. Further alternatively, each of the hooks 78a and 78b can be rotatable about the suspension axis A individually and independently from other components of the ventilator device 10.

The hooks 78a and 78b are preferably formed in such a way that an operating position of the hooks 78a and 78b exists in which a section proceeding from the pivot joint of the hooks 78a and 78b to the respective hook aperture, and preferably also a curved section of the hook aperture adjoining thereto, proceeds in parallel to a section of arms 46a and/or 46b respectively which likewise goes out from the suspension axis A. In FIG. 4 this operating position is the starting position of the hooks 78a and 78b labelled with 78a″ or 78b″ respectively.

The position labelled with 78a′ and/or with 781D′ respectively can be an intermediate position in which the ventilator device 10 suspended from the external structure 79 is subjected by the force of gravity to a supporting moment against a wall situated near the external structure 79 but on the other side with respect to the ventilator device 10. In this case, due only to its own weight the ventilator device 10 supported against the wall situated behind the external structure 79 takes up a stable position in which it can be handled and operated.

The position labelled with 78a and/or with 78b respectively can be an end position in which the ventilator device 10 suspended from the external structure 79 can be suspended freely swinging through the force of gravity, for instance if no wall is available in the vicinity of the external structure 79 to support the ventilator device 10. The ventilator device 10 can be handled and operated when suspended in the end position, too.

The hooks 78a and 78b can be pre-tensioned in the starting position through a spring arrangement. The spring arrangement can be arranged in the second frame section 46, for instance in one of the sleeve sections between a hook 78a or 78b and the carrying handle 52. The three mentioned positions can be the sole latch positions of the hooks 78a and 78b or there can be provided further latch positions of the hooks 78a and 78b about the arrangement axis A.

The suspension arrangement 78 shown in FIG. 4 can also be arranged at the frame 42, in particular at the second frame section 46, without rotatability of the overall arrangement 45 about the axis of rotation D, in particular without relative rotatability of the overall arrangement 45 relative to the second frame section 46.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-15. (canceled)

16. A ventilator device for at least supportive artificial respiration of patients, comprising:
A device housing,
A functional arrangement accommodated in the device housing, where the functional arrangement as functional units exhibits at least a section of a respiratory gas line, a pressure modification device for modifying a respiratory gas pressure in the respiratory gas line, and a control device for controlling the operation at least of the pressure modification device, and
An input/output device arranged at the device housing, accessible from outside the device housing for its operation, for the input of data and/or control commands into the control device and/or for the output of data and information, where the input/output device is linked with the control device for signal transmission,
wherein the ventilator device exhibits a frame, where the device housing with the input/output device arranged thereon is accommodated at the frame rotatably about a virtual axis of rotation.

17. The ventilator device according to claim 16, wherein the device housing exhibits a prism shape which extends along a prism axis with a housing casing wall encircling the prism axis at a radial distance from the prism axis, where the axis of rotation proceeds in parallel to or collinearly with the prism axis.

18. The ventilator device according to claim 16, wherein the axis of rotation proceeds at a distance from the input/output device.

19. The ventilator device according to claim 16, wherein the axis of rotation penetrates through the device housing.

20. The ventilator device according to claim 17, wherein a plurality of physical functional interfaces, which are provided at the device housing, are configured on at least one axial, with respect to the prism axis, end-side front face of the device housing.

21. The ventilator device according to claim 17, wherein a respiratory gas aspiration aperture and/or a connector for establishing an electrical connection and/or a connector for establishing a fluid-mechanical connection and/or a connector for establishing a mechanical connection, which are provided at the device housing, are configured on at least one axial, with respect to the prism axis, end-side front face of the device housing.

22. The ventilator device according to claim 20, wherein the plurality of physical functional interfaces are configured and arranged at the at least one axial end-side front face of the device housing in such a way that with respect to the prism axis they face in the axial direction and/or are connectable with a counter-interface through a connecting movement which takes place in the axial direction.

23. The ventilator device according to claim 16, wherein the frame exhibits a first frame section holding the device housing and a second frame section holding the first frame section, where the first frame section is mounted on the second frame section rotatably about the axis of rotation.

24. The ventilator device according to claim 23, wherein the first frame section is accommodated detachably as specified at the second frame section.

25. The ventilator device according to claim 24, wherein both the first frame section and the second frame section each exhibit an attachment formation which is configured to interact with an attachment counter-formation arranged or configured at the device housing for attachment to the respective frame section.

26. The ventilator device according to claim 16, wherein the frame exhibits a carrying handle configured for manual handling.

27. The ventilator device according to claim 26, wherein the carrying handle proceeds in parallel to the axis of rotation.

28. The ventilator device according to claim 16, wherein the ventilator device includes a suspension arrangement which is configured to fix the ventilator device detachably onto an associated external structure.

29. The ventilator device according to claim 28, wherein the suspension arrangement is movable relative to the device housing and/or relative to the frame.

30. The ventilator device according to claim 29, wherein the suspension arrangement is rotatable about a suspension axis.

31. The ventilator device according to claim 28, wherein the suspension arrangement comprises at least one hook.

32. The ventilator device according to claim 31, wherein the at least one hook is arranged at the frame rotatably about a suspension axis which is parallel to the axis of rotation.

33. A ventilator device for at least supportive artificial respiration of patients, comprising a suspension arrangement which is configured to fix the ventilator device detachably onto an associated external structure, the suspension arrangement being movable relative to the device housing and/or relative to the frame.

34. The ventilator device according to claim 33, wherein the suspension arrangement comprises at least one hook.

35. The ventilator device according to claim 34, wherein the at least one hook is arranged at the frame rotatably about a suspension axis which is parallel to the axis of rotation.
